(19) United States
(12) Patent Application Publication
(10) Pub. No.: US 2019/0008598 A1
(43) Pub. Date: Jan. 10, 2019

(54) FULLY AUTONOMIC ARTIFICIAL INTELLIGENCE ROBOTIC SYSTEM

(71) Applicant: M.S.T. Medical Surgery Technologies Ltd., Yoqneam (IL)

(72) Inventors: Motti Frimer, Zichron Yaakov (IL); Tal Nir, Haifa (IL); Gal Atarot, Kfar Saba (IL)

(73) Assignee: M.S.T. Medical Surgery Technologies Ltd., Yoqneam (IL)

(21) Appl. No.: 16/060,289

(22) PCT Filed: Dec. 6, 2016
(86) PCT No.: PCT/IL2016/051308

§ 371 Date: Jun. 7, 2018

Related U.S. Application Data
(60) Provisional application No. 62/336,672, filed on May 15, 2016.
(60) Provisional application No. 62/290,963, filed on Feb. 4, 2016.
(60) Provisional application No. 62/263,749, filed on Dec. 7, 2015.

(51) Int. Cl. A61B 34/32; A61B 5/06; A61B 5/00; A61B 5/0205; G16H 50/20

(52) U.S. Cl. CPC A61B 34/32; A61B 5/061; A61B 5/0095; A61B 5/02055; A61B 2090/064; A61B 5/4893; A61B 5/7405; A61B 5/7455; G16H 50/20; A61B 5/489

(57) ABSTRACT

The present invention provides a system for identifying at least one surgical procedure, comprising: a. at least one robotic manipulator connectable to said at least one surgical tool; b. at least one imaging device configured to real time provide at least one image in a field of view of a surgical environment; c. processor in communication with said robotic manipulator and said imaging device; and, d. communicable database configured to (i) store said surgical procedure; said at least one surgical procedure is characterized by at least one spatial position of at least one item SP stored; (ii) real-time store at least one of said spatial position, SP item, of at least one said item; wherein said at least one processor is configured to identify at least one said surgical procedure being performed by identifying at least partial match between said SP item and said SP stored

### FIELD OF THE INVENTION

The present invention generally pertains to a system and method for providing autonomic control of surgical tools.

### BACKGROUND OF THE INVENTION

Present systems of control of surgical tools in a surgical field require either manual control or provide slaved responses to an operator's movements, where the movements of the slaved surgical tool reproduce the movements of the operator.

However, this provides a heavy load on an operator, especially where a manual assistant is unskilled or insufficiently skilled, or where the operator must carry our all control alone.

It is therefore a long felt need to provide system which can autonomically identify a surgical procedure from analysis of tool movement, as determined from analysis of at least one image in a field of view.

### SUMMARY OF THE INVENTION

It is an object of the present invention to disclose a system and method for autonomously identifying at least one surgical procedure from analysis of tool movement, as determined from analysis of at least one image in a field of view.

It is another object of the present invention to disclose a system for identifying at least one surgical procedure, comprising:
a. at least one robotic manipulator connectable to said at least one surgical tool;
b. at least one imaging device configured to real time provide at least one image in a field of view of a surgical environment;
c. at least one processor in communication with said robotic manipulator and said imaging device; said processor is configured to control maneuvering of said at least one surgical tool by said robotic manipulator; said at least one processor is configured to (i) analyze at least one image from said at least one imaging device and, (ii) to identify from said at least one image at least one spatial position of at least one item, SPitem; and,
d. at least one communicable database configured to (i) store said at least one surgical procedure; said at least one surgical procedure is characterized by at least one spatial position of at least one item SP_{stored}; (ii) real-time store at least one of said spatial position, SPᵢₜₑₘ, of at least one said item;
wherein said at least one processor is configured to identify at least one said surgical procedure being performed by identifying at least partial match between at least one of said SPᵢₜₑₘ and at least one of said SP_{stored}.

It is another object of the present invention to disclose the system as described above, wherein said item is selected from a group consisting of: said at least one surgical tool, a light source, a blood vessel, an organ, a nerve, and a ligament, a lesion, a tumor, smoke, fluid flow, bleeding, a fixed point, a critical point, and any combination thereof.

It is another object of the present invention to disclose the system as described above, additionally configured to control execution of said procedure by maneuvering said at least one robotic manipulator.

It is another object of the present invention to disclose the system as described above, wherein said processor is additionally configured to control operation of at least one second surgical tool.

It is another object of the present invention to disclose the system as described above, wherein said control of operation of said at least one second surgical tool is autonomic control.

It is another object of the present invention to disclose the system as described above, wherein said at least one second surgical tool is selected from a group consisting of: a laparoscope, an endoscope, an ablator, a fluid supply mechanism, a retractor, a grasper, a suturing mechanism, a pair of tweezers, a forceps, a light source, a vacuum source, a suction device, and any combination thereof.

It is another object of the present invention to disclose the system as described above, wherein said second surgical tool is said surgical tool.

It is another object of the present invention to disclose the system as described above, wherein said spatial position is selected from a group consisting of: a 2D position of at least a portion of the object; a 2D orientation of at least a portion of the object; a 3D position of at least a portion of the object; a 3D orientation of at least a portion of the object; a 2D projection of a 3D position of at least a portion of the object; and any combination thereof.

It is another object of the present invention to disclose the system as described above, additionally configured to provide a message, said message configured to provide information.

It is another object of the present invention to disclose the system as described above, wherein said message is selected from a group consisting of: an audible message, a visual message, a tactile message and any combination thereof.

It is another object of the present invention to disclose the system as described above, wherein said visual message is selected from a group consisting of: a constant-brightness light, a variable-brightness light, a constant-color light, a variable-color light, a patterned light and any combination thereof.

It is another object of the present invention to disclose the system as described above, wherein said audible message is selected from a group consisting of: a constant-loudness sound, a variable-loudness sound, a constant-pitch sound, a variable-pitch sound, a patterned sound and any combination thereof.

It is another object of the present invention to disclose the system as described above, wherein said tactile message is selected from a group consisting of: a vibration, a stationary pressure, a moving pressure and any combination thereof.

It is another object of the present invention to disclose the system as described above, wherein said procedure is initiatable by a member of a group consisting of: manually by a command from an operator, automatically by a command from said processor and any combination thereof.

It is another object of the present invention to disclose the system as described above, additionally configured to accept definition of a location for a member of a group consisting of: said fixed point, said critical point and any combination thereof, and to store said location.

It is another object of the present invention to disclose the system as described above, wherein said procedure is identifiable from at least two fixed points, said procedure comprising a member of a group consisting of: maneuvering said robotic manipulator along a path joining said at least two fixed points, controlling operation of at least one said surgical tool and any combination thereof.

It is another object of the present invention to disclose the system as described above, wherein said database is configured to store a member of a group consisting of: an autonomically-executed procedure, an automatically-executed procedure, a manually executed procedure and any combination thereof.

It is another object of the present invention to disclose the system as described above, wherein at least one record is selectable based upon an identifier.

It is another object of the present invention to disclose the system as described above, wherein said database is configured to store said identifier selected from a group consisting of: an identifier of an operator, an identifier of an operating room, a physical characteristic of an operating room, a number of times said operator has executed a procedure, a physical characteristic of an operating room, a date of said procedure, a time of said procedure, a duration of said procedure, a vital sign of a patient, an identifier of a patient, a physical characteristic of a patient, an outcome of a procedure, length of hospital stay for a patient, a readmission for a patient a datum from a patient's medical history, number of said at least one procedures carried out by an operator, cleaning status of an operating room, a general datum, and any combination thereof.

It is another object of the present invention to disclose the system as described above, wherein said datum from a patient's medical history is selected from a group consisting of: an illness, an outcome of an illness, a previous procedure, an outcome of a previous procedure, a genetic factor, an effect on said patient of said genetic factor, a predicted effect on said patient of said genetic factor, a medical treatment, an allergy, a medical condition, a psychological factor, and any combination thereof, an outcome of a procedure, length of hospital stay for a patient, a readmission for a patient, a medical treatment for a patient, a subsequent procedure, number of subsequent procedures carried out an operator, a general datum, and any combination thereof.

It is another object of the present invention to disclose the system as described above, wherein said cleaning status of an operating room is selected from a group consisting of: time of last cleaning, date of last cleaning, cleaning procedure, cleaning material

It is another object of the present invention to disclose the system as described above, wherein said outcome is selected from a group consisting of: a successful aspect, a partially successful aspect, a partial failure in an aspect, a complete failure in an aspect, and any combination thereof.

It is another object of the present invention to disclose the system as described above, wherein said aspect is selected from a group consisting of: a complication during a procedure, a complication during another procedure, a component where recovery is smooth and uncomplicated, a rate of recovery from a procedure, a rate of recovery from a complication, a long-term effect of a procedure, a long-term effect of a complication, amount of bleeding during a procedure, amount of bleeding during another procedure, return of an abnormality, speed of healing, an adhesion, patient discomfort, and any combination thereof.

It is another object of the present invention to disclose the system as described above, wherein said general datum is selected from a group consisting of: an image of at least a portion of a surgical field, an identifier of an operator, a rating for an operator, a physical characteristic of a patient, a physical characteristic of an operating room, an identifier of a procedure, type of procedure, time of a beginning of a procedure, time of an intermediate point of a procedure, time of an end point of a procedure, duration of a procedure, time between end of a procedure and beginning of another procedure, time of creation of a critical point, location of a critical point, time of creation of a fixed point, location of a fixed point, a medication, a medical device, an identifier of a surgical object, a type of a surgical object, a number used for a surgical object, a cleaning status for a surgical object, a comment, a parameter, a metric, occurrence of a malfunction, severity of a malfunction, start time of a malfunction, end time of a malfunction, reason for start of a malfunction, reason for end of a malfunction, occurrence of an adverse event, a test, an image from another modality, an overlay, a label, a note, and any combination thereof.

It is another object of the present invention to disclose the system as described above, wherein said parameter is selected from a group consisting of: 2D position of at least a portion of at least one item, 2D orientation of at least a portion of at least one item, 3D position of at least a portion of at least one item, 3D orientation of at least a portion of at least one item, 2D projection of a 3D position of at least a portion of said at least one item, movement of at least a portion of at least one said item, energy use; idle time, approach time, speed, maximum speed, speed profile, acceleration, motion smoothness, path length, distance efficiency, depth perception, transit profile, deviation on horizontal plane, deviation on vertical plane, response orientation, economy of area (EOA), economy of volume (EOV), number of movements, force range, interquartile force range, integral of the force, integral of the grasping force, integral of the Cartesian force, first derivative of the force, second derivative of the force, third derivative of the force, lighting level, amount of suction, amount of fluid flow, heating level in an ablator, amount of defogging, amount of smoke removal, activation of an item, deactivation of an item, bleeding, change in heart rate, change in blood pressure, change in color of an organ,

It is another object of the present invention to disclose the system as described above, wherein said physical characteristic of an operating room is selected from a group consisting of: temperature, humidity, type of lighting, and any combination thereof.

It is another object of the present invention to disclose the system as described above, wherein said medical device is selected from a group consisting of: a heating blanket, a pump, a catheter, and any combination thereof.

It is another object of the present invention to disclose the system as described above, wherein said occurrence of an adverse event is selected from a group consisting of: unexpected bleeding, undesirable change in blood pressure, undesirable change in heart rate, undesirable change in consciousness state, pain, and any combination thereof.

It is another object of the present invention to disclose the system as described above, wherein said physical characteristic of said patient is selected from a group consisting of: age, height, weight, body mass index, physical parameter of said patient, and any combination thereof.

It is another object of the present invention to disclose the system as described above, wherein said physical parameter of said patient is selected from a group consisting of: health status, blood pressure, heart rate, blood gasses, blood volume, blood hemoglobin, breathing rate, breath depth, EEG, ECG, sweating, and any combination thereof,

It is another object of the present invention to disclose the system as described above, wherein said medication is selected from a group consisting of: an antibiotic, an anesthetic, plasma, blood, saline, coagulant, anticoagulant, blood pressure medication, heart medication, and any combination thereof.

It is another object of the present invention to disclose the system as described above, wherein said medical treatment is selected from a group consisting of: administering a medication, applying a medical device, prescribing a course of exercise, administering physiotherapy, and any combination thereof.

It is another object of the present invention to disclose the system as described above, wherein said test is selected from a group consisting of: a blood test, a blood pressure measurement, an EEG, and ECG, and any combination thereof.

It is another object of the present invention to disclose the system as described above, wherein said other modality is selected from a group consisting of: MRI, CT, ultrasound, X-ray, fluorography, fluoroscopy, molecular imaging, scintigraphy, SPECT, positron emission tomography (PET), other types of tomography, elastography, tactile imaging, photoacoustic imaging, thermography, functional near-infrared spectroscopy (FNIR) and any combination thereof.

It is another object of the present invention to disclose the system as described above, wherein said image from said other modality can be stored or real-time.

It is another object of the present invention to disclose the system as described above, wherein a member of a group consisting of said note, said comment and any combination thereof is selected from a group consisting of: a descriptor of a previously-performed procedure, a list of at least one previously performed procedure, how a procedure was executed, why a procedure was chosen, an assessment of a patient, a prediction, an item to be added to a medical history, a method of executing a procedure, and any combination thereof.

It is another object of the present invention to disclose the system as described above, wherein said critical point is selected from a group consisting of: a location in said surgical field, a beginning of a procedure, an end of a procedure, an intermediate point in a procedure and any combination thereof.

It is another object of the present invention to disclose the system as described above, wherein a member of a group consisting of said at least one image of at least a portion of a surgical field, said second modality image and any combination thereof is selected from a group consisting of: a 2D image, a 3D image, a panoramic image, a high resolution image, a 3D image reconstructed from at least one 2D image, a 3D stereo image, and any combination thereof.

It is another object of the present invention to disclose the system as described above, wherein said identifier is storable as a function of time.

It is another object of the present invention to disclose the system as described above, wherein said system is in at least one-way communication with a member of a group consisting of: digital documentation, PACS, navigation, a health IT system, and any combination thereof.

It is another object of the present invention to disclose the system as described above, wherein said communication comprises communicating procedure-related data selected from a group consisting of: a name of an operator, a name of an assistant, an identifier of an operator, an identifier of an assistant, an identifier of an operating room, a physical characteristic of an operating room, a physical characteristic of an operating room as a function of time, a date of said procedure, a start time of said procedure, an end time of said procedure, a duration of said procedure, a vital sign of a patient, a name of a patient, a physical characteristic of a patient, an outcome of said procedure, length of hospital stay for a patient, a readmission for a patient, a number of times said operator has executed a procedure, a date of a previous procedure, a start time of a previous procedure, an end time of a previous procedure, a duration of a previous procedure, a vital sign of a previous patient, a name of a previous patient, a physical characteristic of a previous patient, length of hospital stay for a previous patient, a readmission for a previous patient, and any combination thereof.

It is another object of the present invention to disclose the system as described above, wherein said selection of said at least one surgical procedure is at least partially based on at least one procedure-related datum.

It is another object of the present invention to disclose the system as described above, further comprising a manual override, said procedure stoppable by means of said manual override.

It is another object of the present invention to disclose a method for identifying at least one surgical procedure, comprising steps of:
a. providing a system for identifying at least one surgical procedure comprising:
   i. at least one robotic manipulator connectable to said at least one surgical tool;
   ii. at least one imaging device configured to real time provide at least one image in a field of view of a surgical environment;
   iii. at least one processor in communication with said robotic manipulator and said imaging device, said processor is configured to control maneuvering of said at least one surgical tool by said robotic manipulator; said at least one processor is configured to (i) analyze at least one image from said at least one imaging device and (ii) identify from said at least one image at least one spatial position of at least one item, SPᵢₜₑₘ; and
   iv. at least one communicable database configured to (i) store at least one surgical procedure; said at least one surgical procedure is characterized by at least one spatial position of at least one item SP_{stored}; real-time store at least one said spatial position, SPitem, of at least one said item;
b. connecting said at least one surgical tool to said robotic manipulator;
c. acquiring, via said imaging device, at least one said image of said field of view;
d. analyzing said at least one image and identifying, from said analysis, said at least one spatial position of said at least one item, SPitem;
e. real-time storing at least one said spatial position of at least one said item, SPitem;
and f. identifying at least one said surgical procedure being performed by identifying at least partial match between at least one of said SPᵢₜₑₘ and at least one of said SP_{stored}.

It is another object of the present invention to disclose the method as described above, additionally comprising step of selecting said item from a group consisting of: said at least one surgical tool, a light source, a blood vessel, an organ, a nerve, and a ligament, a lesion, a tumor, smoke, fluid flow, bleeding, a fixed point, a critical point, and any combination thereof.

It is another object of the present invention to disclose the method as described above, additionally comprising step of controlling execution of said at least one procedure by maneuvering said at least one robotic manipulator.

It is another object of the present invention to disclose the method as described above, additionally comprising step of controlling operation of at least one second surgical tool.

It is another object of the present invention to disclose the method as described above, additionally comprising step of autonomically controlling operation of said at least one second surgical tool.

It is another object of the present invention to disclose the method as described above, additionally comprising step of selecting said at least one second surgical tool from a group consisting of: a laparoscope, an endoscope, a suction device, a vacuum source, a light source, an ablator, a fluid supply mechanism, a retractor, a grasper, a suturing mechanism, a pair of tweezers, a pair of tweezers, a forceps, and any combination thereof.

It is another object of the present invention to disclose the method as described above, additionally comprising step of selecting second surgical tool to be said surgical tool.

It is another object of the present invention to disclose the method as described above, additionally comprising step of selecting said spatial position from a group consisting of: a 2D position of at least a portion of the object; a 2D orientation of at least a portion of the object; a 3D position of at least a portion of the object; a 3D orientation of at least a portion of the object; a 2D projection of a 3D position of at least a portion of the object; and any combination thereof.

It is another object of the present invention to disclose the method as described above, additionally comprising step of providing a message, said message configured to provide information.

It is another object of the present invention to disclose the method as described above, additionally comprising step of selecting said message from a group consisting of: an audible message, a visual message, a tactile message and any combination thereof.

It is another object of the present invention to disclose the method as described above, additionally comprising step of selecting said visual message from a group consisting of: a constant-brightness light, a variable-brightness light, a constant-color light, a variable-color light, a patterned light and any combination thereof.

It is another object of the present invention to disclose the method as described above, additionally comprising step of selecting said audible message from a group consisting of: a constant-loudness sound, a variable-loudness sound, a constant-pitch sound, a variable-pitch sound, a patterned sound and any combination thereof.

It is another object of the present invention to disclose the method as described above, additionally comprising step of selecting said tactile message from a group consisting of: a vibration, a stationary pressure, a moving pressure and any combination thereof.

It is another object of the present invention to disclose the method as described above, additionally comprising step of initiating said procedure by a member of a group consisting of: manually by a command from an operator, automatically by a command from said processor and any combination thereof.

It is another object of the present invention to disclose the method as described above, additionally comprising steps of accepting definition of a location of a member of a group consisting of: said fixed point, said critical point and any combination thereof, and of storing said location.

It is another object of the present invention to disclose the method as described above, additionally comprising step of identifying said procedure from said at least two fixed points, said procedure comprising a member of a group consisting of: maneuvering said robotic manipulator along a path joining said at least two fixed points, controlling operation of at least one said surgical tool along a path joining said at least two fixed points and any combination thereof.

It is another object of the present invention to disclose the method as described above, additionally comprising step of storing in said database a member of a group consisting of: an autonomically-executed procedure, an automatically-executed procedure, a manually executed procedure and any combination thereof.

It is another object of the present invention to disclose the method as described above, additionally comprising step of selecting at least one record based upon an identifier.

It is another object of the present invention to disclose the method as described above, additionally comprising steps of selecting said identifier from a group consisting of: an identifier of an operator, an identifier of an operating room, a physical characteristic of an operating room, a number of times said operator has executed a procedure, a physical characteristic of an operating room, a date of said procedure, a time of said procedure, a duration of said procedure, a vital sign of a patient, an identifier of a patient, a physical characteristic of a patient, an outcome of a procedure, length of hospital stay for a patient, a readmission for a patient a datum from a patient's medical history, number of said at least one procedures carried out by an operator, cleaning status of an operating room, a general datum, and any combination thereof; and of storing in said database and of storing said identifier in said database.

It is another object of the present invention to disclose the method as described above, additionally comprising step of selecting said datum from a patient's medical history from a group consisting of: an illness, an outcome of an illness, a previous procedure, an outcome of a previous procedure, a genetic factor, an effect on said patient of said genetic factor, a predicted effect on said patient of said genetic factor, a medical treatment, an allergy, a medical condition, a psychological factor, and any combination thereof, an outcome of a procedure, length of hospital stay for a patient, a readmission for a patient, a medical treatment for a patient, a subsequent procedure, number of subsequent procedures carried out an operator, a general datum, and any combination thereof.

It is another object of the present invention to disclose the method as described above, additionally comprising step of selecting said cleaning status of an operating room from a group consisting of: time of last cleaning, date of last cleaning, cleaning procedure, cleaning material

It is another object of the present invention to disclose the method as described above, additionally comprising step of selecting said outcome from a group consisting of: a successful aspect, a partially successful aspect, a partial failure in an aspect, a complete failure in an aspect, and any combination thereof.

It is another object of the present invention to disclose the method as described above, additionally comprising step of selecting said aspect from a group consisting of: a complication during a procedure, a complication during another procedure, a component where recovery is smooth and uncomplicated, a rate of recovery from a procedure, a rate of recovery from a complication, a long-term effect of a procedure, a long-term effect of a complication, amount of bleeding during a procedure, amount of bleeding during another procedure, return of an abnormality, speed of healing, an adhesion, patient discomfort, and any combination thereof.

It is another object of the present invention to disclose the method as described above, additionally comprising step of selecting said general datum from a group consisting of: an image of at least a portion of a surgical field, an identifier of an operator, a rating for an operator, a physical characteristic of a patient, a physical characteristic of an operating room, an identifier of a procedure, type of procedure, time of a beginning of a procedure, time of an intermediate point of a procedure, time of an end point of a procedure, duration of a procedure, time between end of a procedure and beginning of another procedure, time of creation of a critical point, location of a critical point, time of creation of a fixed point, location of a fixed point, a medication, a medical device, an identifier of a surgical object, a type of a surgical object, a number used for a surgical object, a cleaning status for a surgical object, a comment, a parameter, a metric, occurrence of a malfunction, severity of a malfunction, start time of a malfunction, end time of a malfunction, reason for start of a malfunction, reason for end of a malfunction, occurrence of an adverse event, a test, an image from another modality, an overlay, a label, a note, and any combination thereof.

It is another object of the present invention to disclose the method as described above, additionally comprising step of selecting said parameter from a group consisting of: 2D position of at least a portion of at least one item, 2D orientation of at least a portion of at least one item, 3D position of at least a portion of at least one item, 3D orientation of at least a portion of at least one item, 2D projection of a 3D position of at least a portion of said at least one item, movement of at least a portion of at least one said item, energy use; idle time, approach time, speed, maximum speed, speed profile, acceleration, motion smoothness, path length, distance efficiency, depth perception, transit profile, deviation on horizontal plane, deviation on vertical plane, response orientation, economy of area (EOA), economy of volume (EOV), number of movements, force range, interquartile force range, integral of the force, integral of the grasping force, integral of the Cartesian force, first derivative of the force, second derivative of the force, third derivative of the force, lighting level, amount of suction, amount of fluid flow, heating level in an ablator, amount of defogging, amount of smoke removal, activation of an item, deactivation of an item, bleeding, change in heart rate, change in blood pressure, change in color of an organ,

It is another object of the present invention to disclose the method as described above, additionally comprising step of selecting said physical characteristic of an operating room from a group consisting of: temperature, humidity, type of lighting, and any combination thereof.

It is another object of the present invention to disclose the method as described above, additionally comprising step of selecting said medical device is selected from a group consisting of: a heating blanket, a pump, a catheter, and any combination thereof.

It is another object of the present invention to disclose the method as described above, wherein said occurrence of an adverse event from a group consisting of: unexpected bleeding, undesirable change in blood pressure, undesirable change in heart rate, undesirable change in consciousness state, pain, and any combination thereof.

It is another object of the present invention to disclose the method as described above, additionally comprising step of selecting said physical characteristic of said patient from a group consisting of: age, height, weight, body mass index, physical parameter of said patient, and any combination thereof.

It is another object of the present invention to disclose the method as described above, additionally comprising step of selecting said physical parameter of said patient from a group consisting of: health status, blood pressure, heart rate, blood gasses, blood volume, blood hemoglobin, breathing rate, breath depth, EEG, ECG, sweating, and any combination thereof,

It is another object of the present invention to disclose the method as described above, additionally comprising step of selecting said medication from a group consisting of: an antibiotic, an anesthetic, plasma, blood, saline, coagulant, anticoagulant, blood pressure medication, heart medication, and any combination thereof.

It is another object of the present invention to disclose the method as described above, additionally comprising step of selecting said medical treatment from a group consisting of: administering a medication, applying a medical device, prescribing a course of exercise, administering physiotherapy, and any combination thereof.

It is another object of the present invention to disclose the method as described above, additionally comprising step of selecting said test from a group consisting of: a blood test, a blood pressure measurement, an EEG, and ECG, and any combination thereof.

It is another object of the present invention to disclose the method as described above, additionally comprising step of selecting said other modality from a group consisting of: MRI, CT, ultrasound, X-ray, fluorography, fluoroscopy, molecular imaging, scintigraphy, SPECT, positron emission tomography (PET), other types of tomography, elastography, tactile imaging, photoacoustic imaging, thermography, functional near-infrared spectroscopy (FNIR) and any combination thereof.

It is another object of the present invention to disclose the method as described above, wherein said image from said other modality can be stored or real-time.

It is another object of the present invention to disclose the method as described above, additionally comprising step of selecting a member of a group consisting of said note, said comment and any combination thereof from a group consisting of: a descriptor of a previously-performed procedure, a list of at least one previously performed procedure, how a procedure was executed, why a procedure was chosen, an assessment of a patient, a prediction, an item to be added to a medical history, a method of executing a procedure, and any combination thereof.

It is another object of the present invention to disclose the method as described above, additionally comprising step of selecting said critical point from a group consisting of: a location in said surgical field, a beginning of a procedure, an end of a procedure, an intermediate point in a procedure and any combination thereof.

It is another object of the present invention to disclose the method as described above, additionally comprising step of selecting a member of a group consisting of said at least one image of at least a portion of a surgical field, said second modality image and any combination thereof from a group consisting of: a 2D image, a 3D image, a panoramic image, a high resolution image, a 3D image reconstructed from at least one 2D image, a 3D stereo image, and any combination thereof.

It is another object of the present invention to disclose the method as described above, additionally comprising step of storing said identifier as a function of time.

It is another object of the present invention to disclose the method as described above, additionally comprising step of providing for said system at least one-way communication with a member of a group consisting of: digital documentation, PACS, navigation, a health IT system, and any combination thereof.

It is another object of the present invention to disclose the method as described above, additionally comprising steps of providing, for said communication, procedure-related data, and of selecting said procedure-related data from a group consisting of: a name of an operator, a name of an assistant, an identifier of an operator, an identifier of an assistant, an identifier of an operating room, a physical characteristic of an operating room, a physical characteristic of an operating room as a function of time, a date of said procedure, a start time of said procedure, an end time of said procedure, a duration of said procedure, a vital sign of a patient, a name of a patient, a physical characteristic of a patient, an outcome of said procedure, length of hospital stay for a patient, a readmission for a patient, a number of times said operator has executed a procedure, a date of a previous procedure, a start time of a previous procedure, an end time of a previous procedure, a duration of a previous procedure, a vital sign of a previous patient, a name of a previous patient, a physical characteristic of a previous patient, length of hospital stay for a previous patient, a readmission for a previous patient, and any combination thereof.

It is another object of the present invention to disclose the method as described above, additionally comprising step of said selection of said at least one surgical procedure is at least partially based on at least one procedure-related datum.

It is another object of the present invention to disclose the method as described above, additionally comprising steps of providing a manual override, and of stopping said procedure by means of said manual override.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1A-B schematically illustrates control of a laparoscope in the prior art; and

FIG. 2A-B schematically illustrates control of a laparoscope in the present invention.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for autonomously identifying at least one surgical procedure from analysis of tool movement, as determined from analysis of at least one image in a field of view.

The term “automatic procedure” hereinafter refers to a procedure in which one surgical tool automatically responds to a movement or other action of a second surgical tool. Non-limiting examples of an automatically-executed procedure includes tracking a surgical tool by an endoscope or changing a lighting level in response to an increase in a perceived amount of smoke.

The term “autonomic procedure” hereinafter refers to a procedure which can be executed independently of actions of a surgeon or of other tools. Non-limiting examples of autonomic procedures include executing a complete suture and executing a plurality of sutures to close an incision.

The term “fixed point” hereinafter refers to a point in 3D space which is fixed relative to a known location. The known location can be for non-limiting example, an insertion point, a known location in or on a patient, a known location in an environment around a patient (e.g., an attachment point of a robotic manipulator to an operating table, a hospital bed, or the walls of a room), or a known location in a manipulation system, a practice dummy, or a demonstrator.

The term “item” hereinafter refers to any identifiable thing within a field of view of an imaging device. An item can be something belonging to a body or a medical object introducible into a body. An item can also comprise a thing such as, for non-limiting example, shrapnel or parasites, a non-physical thing such as a fixed point or a critical point, a physical thing such as smoke, fluid flow, bleeding, dirt on a lens, lighting level, etc.

The term “object” hereinafter refers to an item naturally found within a body cavity. Non-limiting examples of an object include a blood vessel, an organ, a nerve, and a ligament, as well as an abnormality such as a lesion and a tumor.

The term “tool” or “surgical tool” hereinafter refers to an item mechanically introducible into a body cavity. Non-limiting examples of a tool include a laparoscope, an endoscope, a light, a suction device, a grasper, a suture material, a needle, and a swab.

The term “surgical object” hereinafter refers to a surgical tool, a robotic manipulator or other maneuvering system configured to manipulate a surgical tool, at least a portion of a light source, and at least a portion of an ablator.

The term “operator” hereinafter refers to any of: a principal operator such as, but not limited to, the surgeon carrying out the main parts of the procedure, an assistant such as, but not limited to, a nurse and an observer such as, but not limited to, a senior surgeon providing instruction to or assessing a principal operator. An identifier for an operator can include, but is not limited to, a name, an ID number, a function and any combination thereof.

The term “identifiable unit” hereinafter refers to an identifiable purposive activity during a surgical operation, typically a minimal identifiable activity. Examples include, but are not limited to, movement of a needle and forceps to the site where a suture is to be made, making a knot in suture thread, activating fluid flow, and making an incision.

The term “surgical task” hereinafter refers to a connected series of at least one identifiable unit which comprises an identifiable activity. Non-limiting examples of surgical tasks that comprise more than one identifiable unit include, but are not limited to, making one suture, removing incised tissue from a surgical field, and clearing debris from a surgical field. A non-limiting example of a surgical task that comprises a single identifiable unit is making an incision.

The term “complete procedure” hereinafter refers to a connected series of at least one surgical task which forms an independent unit. For non-limiting example, closing an incision with one or more sutures will be referred to as a complete procedure.

The term “procedure” or “surgical procedure” hereinafter refers to at least a portion of a surgical operation, with the portion of the surgical operation including at least one identifiable unit. For non-limiting example, in increasing order of complexity, a procedure can comprise tying the knot in a suture, making a single suture, or closing an incision with a series of sutures.

The term “automatic procedure” hereinafter refers to a procedure in which one surgical tool automatically responds to a movement or other action of a second surgical tool. Non-limiting examples of an automatically-executed procedure includes tracking a surgical tool by an endoscope or changing a lighting level in response to an increase in a perceived amount of smoke.

The term “autonomic procedure” hereinafter refers to a procedure which can be executed independently of actions of a surgeon or of other tools. Non-limiting examples of autonomic procedures include executing a complete suture and executing a plurality of sutures to close an incision.

The term “about” hereinafter refers to a range of 25% around the quoted number.

The system of the present invention comprises a system for disclose a system and method for autonomously identifying, during a surgical procedure such as a laparoscopic procedure, the nature of at least a portion of the surgical procedure from analysis of tool movement, as determined from analysis of at least one image in a field of view. The heart of the system is an advanced artificial intelligence (AI) system running on at least one processor which is capable of analyzing a scene in a field of view (FOV), as captured in real time by an imaging device and, from the analysis (and possibly from other provided information) forming an understanding of what is occurring. From this understanding, the system derives at least one appropriate procedure, a system procedure, to be carried out under the control of the processor, where the system procedure comprises at least one movement of at least one surgical tool. In some embodiments, the system procedure will be to assist the surgeon in carrying out his surgical procedure. In some, preferred embodiments, the system procedure will be to autonomically (autonomously) carry out a system procedure without the surgeon's intervention.

The basis of the analysis is a determination of the spatial position and orientation of at least one item in a field of view. The spatial position can be a 2D position (for non-limiting example, in the plane of the field of view) of at least a portion of the item; a 2D orientation (for non-limiting example, in the plane of the field of view) of at least a portion of the item; a 3D position of at least a portion of the item; a 3D orientation of at least a portion of the item; a 2D projection of a 3D position of at least a portion of the item, and any combination thereof.

The movement of the item can be selected from a group consisting of: a maneuver of a surgical object carried out by a robotic manipulator connected to the surgical object, a movement of part of an item, a movement of part of a surgical object, a change in state of a surgical object, and any combination thereof. Non-limiting examples of movement of a surgical object include displacing it, rotating it, zooming it, or, for a surgical object with at least one bendable section, changing its articulation. Non-limiting examples of movements of part of a surgical object are opening or closing a grasper or retractor, or operating a suturing mechanism. Non-limiting examples of a change in state of a surgical object include: altering a lighting level, altering an amount of suction, altering an amount of fluid flow, altering a heating level in an ablator, altering an amount of defogging, or altering an amount of smoke removal.

At least one procedure can be stored in a database in communication with the processor; the procedure can comprise at least one real-time image, at least one identifying tag, and any combination thereof. A stored procedure can be a manually-executed procedure, an automatically-executed procedure, an autonomically-executed procedure and any combination thereof.

For non-limiting example, an analysis of an FOV can indicate that a procedure being executed comprises suturing. In some embodiments, if the analysis shows that suturing is occurring, the system procedure can comprise moving and zooming a laparoscope so as to provide an optimum view of the suturing during all of the stages of suturing, such as, for non-limiting example, zooming in for close work such as making a tie or penetrating tissue, zooming out for an overview during movement from one suture to a next suture, and repositioning so as to keep at least one surgical tool in view as the surgical tool is moved from the location of one suture to the location of a next suture.

In preferred embodiments, a system procedure can autonomically perform the procedure. For non-limiting example, a system can recognize that a next procedure is to perform at least one suture. Under such a scenario, the autonomic procedure to create a suture would comprise moving a suture needle and suture thread to the site of a next suture, inserting the suture needle through the tissue, tying a knot, and clipping the suture thread. In some embodiments, the system procedure can additionally comprise at least one of: moving at least one retractor to allow an incision to at least partially close, moving at least one grasping tool to close an incision, placing at least one grasping tool to hold two portions of tissue in a position, moving or placing a swab or sponge, altering a lighting level, applying suction, applying lavage, moving a needle and the suture thread to the location of a next suture, and positioning a laparoscope to enable the surgeon to observe the system procedure.

In some embodiments, the system also comprises an override mechanism so that the surgeon can stop or alter a system procedure.

In some embodiments, the system can interface with and, preferably, control, other tools, such as, but not limited to, suction devices, lighting, ablators, and fluid suppliers. For non-limiting example, if the system determines that there is effusion of blood from an incision, it could command that a suction device be brought into the region of blood effusion and that suction be applied to the blood.

In some embodiments, the system can interface with and, preferably, control, devices in the operating room environment such as, but not limited to, anesthesia equipment, a surgical table, a surgical table accessory, a surgical boom, a surgical light, a surgical headlight, a surgical light source, a vital signs monitor, an electrosurgical generators, a defibrillator and any combination thereof.

In some embodiments, the system can interface with external software such as, but not limited to, hospital databases, as described hereinbelow.

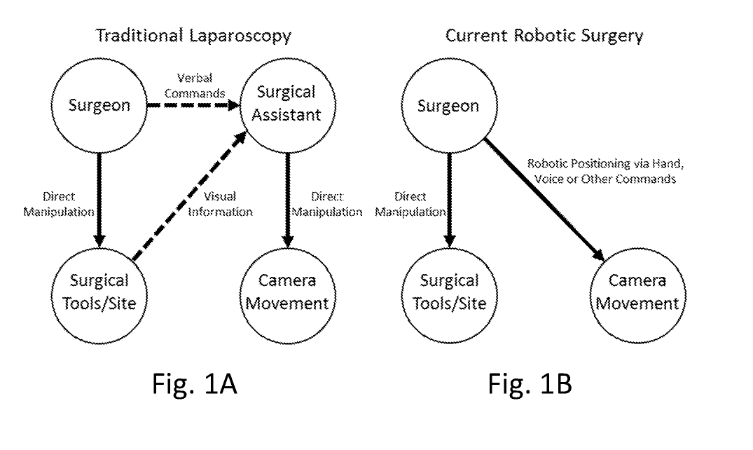

Examples of the flow of control for the laparoscope in the prior art are shown in FIGS. 1A and B. As shown in FIG. 1A, in traditional laparoscopy, a human surgical assistant directs the laparoscope (right vertical solid line). An operator (the surgeon) manipulates tools at the surgical site (left vertical solid arrow). The operator can command the assistant (horizontal dashed arrow) to position the laparoscope, the assistant, from the displayed image and his knowledge of the procedure, can position the laparoscope without command from the operator (diagonal dashed line) and any combination thereof.

FIG. 1B shows a typical flow of control for current robotic systems. In current systems, there is no surgical assistant; all control is carried out by the operator (the surgeon). The operator manipulates tools at the surgical site (vertical solid arrow), and also commands movements of the laparoscope (diagonal solid arrow). Movements of the laparoscope can be commanded by voice, by touching a touchscreen, by manipulating a device, by a predetermined body movement, and any combination thereof.

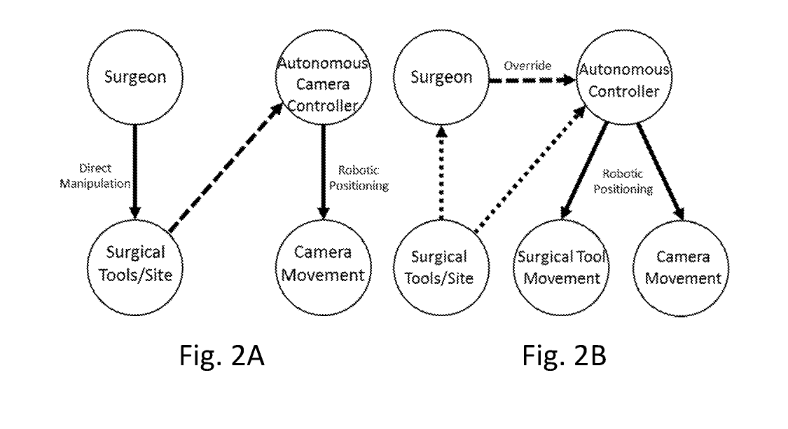

FIG. 2A shows a typical flow of control for some embodiments of the system of the present invention. An operator (the surgeon) manipulates tools at the surgical site (left vertical solid arrow). An autonomous controller, typically camera-controlled, receives information from the surgical tools and/or the surgical site, and, based on the observed information and stored information about the procedure, manipulates the laparoscope (camera). In some embodiments, not shown in the figure, the operator can command the camera controller, by voice, by touching a touchscreen, by manipulating a device, by a predetermined body movement, and any combination thereof.

In preferred embodiments, the system determines the current state of the procedure that is being undertaken and adjusts the camera's/arm's behavior by incorporating preexisting knowledge about the visualization requirements and types of movements needed for the procedure. FIG. 2B shows a flow of control for some embodiments of the present system where the system can autonomously perform a procedure.

In some embodiments, the AI system is capable of analyzing a scene in a field of view and, from the analysis (and possibly from other provided information) forming an understanding of what is occurring. From this understanding, the AI system can predict the next steps in the procedure and can respond appropriately.

In some embodiments, the system can perform at least one procedure independently, autonomically, without an operator's intervention. In some embodiments, the system can perform at least one procedure automatically, such that at least one action of the system is not under the direct control of an operator. In some embodiments, for at least one procedure, at least one action of the system is under manual control of an operator.

Non-limiting examples of control, which can be automatic control, autonomic control and any combination thereof, include: adjusting zoom, including transparently switching between physical zoom and digital zoom; altering FOV, including transparently switching between physically altering a FOV and digitally altering it (e.g., by means of digitally changing a selected portion of an FOV); adjusting lighting level, including turning lighting on or off and maneuvering at least one light source; adjusting fluid flow rate, adjusting suction and any combination thereof.

Non limiting examples of automatic or autonomic control of lighting include: increasing the lighting level if a region of the field of view is undesirably dark, either because of shadowing by a tool or by tissue, or because of failure of a light source; and increasing the lighting level at the beginning of a procedure, such as a suturing, for which a high level of lighting is desirable and decreasing the lighting level at the end of the procedure.

A non-limiting example of automatic control of a tool is control of zooming during suturing so that an operator has, at all times, an optimum view of the suturing. Under automatic control, the laparoscope will be zoomed in during the tying process, zoomed out after a suture has been completed to allow the operator a better view of the site, and will follow the suturing tools as they are moved to the site of the next suture, all without direct intervention by an operator.

A non-limiting example of autonomic functioning of the system is an extension of the above, where the system carries out a suturing process, including moving the suturing tools, tying the sutures and cutting the suture threads, and, in preferred embodiments, moving an imaging device so that the process can be displayed so it can be overseen. In some embodiments of autonomic control of suturing, the system can perform several sutures, so that, once a suturing process is started, either autonomically or by being commanded by an operator, an entire incision will be sutured, with the system moving autonomically from one suturing site to the next.

In preferred embodiments, an override facility is provided, so that the operator can intervene manually. Manual intervention, via a manual override, can occur, for non-limiting example, if an event occurs that requires immediate action.

In some embodiments, the system can have different operation modes, depending on the identified procedure, or the viewed scene.

In preferred embodiments, the system can provide a message for an operator. Typical messages include, but are not limited to: a warning (for non-limiting example, of unusual blood flow, of a change in a vital sign), a suggestion of a procedure to be carried out, a request to start a procedure, a request to identify a fixed point, a suggestion of a location for a fixed point, and any combination thereof.

The message can be an audible message, a visual message, a tactile message and any combination thereof. A visual message can be a constant-brightness light, a variable-brightness light, a constant-color light, a variable-color light, a patterned light and any combination thereof. A non-limiting example of a patterned visual message is a word or phrase. An audible message can be a constant-loudness sound, a variable-loudness sound, a constant-pitch sound, a variable-pitch sound, a patterned sound and any combination thereof. A non-limiting example of a patterned audible message is a spoken word or phrase. A tactile message can be a vibration, a stationary pressure, a moving pressure and any combination thereof. The pressure can be to any convenient position on an operator. Non-limiting examples include a finger, a hand, an arm, a chest, a head, a torso, and a leg.

In some embodiments, the system identifies surgical tools in the working area; in some embodiments, objects such as organs, lesions, bleeding and other items related to the patient are identified, and/or smoke, flowing fluid, and the quality of the lighting (level, dark spots, obscured spots, etc.). If smoke, flowing fluid or bleeding is identified, the system can respond by e.g., virtual smoke or fogging removal (removal of smoke of fogging from an image via software), increasing a lighting level, providing light from an additional direction or angle, starting smoke or fog removal measures such as flowing fluid across a lens or through an area obscured by smoke or fog, starting suction, alerting an operator to the bleeding, clarifying an image either in software or my changing zoom or focus, applying adaptive optics correction, and any combination thereof.

In preferred embodiments, software-based super-resolution techniques can be used to sharpen images without changing zoom or focus. Such super-resolution techniques can be used to seamlessly change to and from physical zoom and software (digital) zoom, and to seamlessly change to and from physically changing an FOV and changing an FOV via software. Software alteration of an FOV can include selection of another portion of an image, software correction of distortion in an image and any combination thereof.

In some embodiments, the system can identify tools in the working area, either by means of image recognition or by means of tags associated with the tools. The tags can comprise color-coding or other mechanical labelling, or electronic coding, such as, but not limited to radiofrequency signals. Radiofrequency signal can be the same for the different tools or they can differ for at least one tool. The system can recognize a labelled tool from its mechanical or radiofrequency coding, a tool can be identified by an operator, and any combination thereof.

In some embodiments, the system can recognize gestures and can respond appropriately to the gestures. The gestures can be related to the action (e.g., recognizing suturing), not related (e.g., crossing tools to indicate that the system is to take a picture of the field of view), and any combination thereof. The response to the gesture can be a fixed response (e.g., taking a picture, zooming in or out) or it can be a flexible response (e.g., adjusting zoom and location of endoscope to provide optimum viewing for a suturing procedure).

In some embodiments, commands can be entered via a touchscreen or via the operator's body movements. The touchscreen can be in a monitor, a tablet, a phone, or any other device comprising a touchscreen and configured to communicate with the system. The body movements can be gestures, eye movements, and any combination thereof. In preferred embodiments, eye movements can be used.

In some embodiments, orientation indications are provided and the horizon is markable. The orientation indication can be based items in the field of view such as organs, on “dead reckoning”, and any combination thereof.

Orientation by dead reckoning can be known by providing a known orientation at a start of a procedure, by entering an orientation at a start of a procedure, by recognition of an orientation marker attached to a patient or to an operating table, and any combination thereof.

In some embodiments, missed tools can be identified and at least one of the operator alerted to the missing tool or the missing tool automatically recognized and automatically labelled.

In some embodiments, control of movement of the surgical tool or laparoscope can include a member of a group consisting of: changing arm movement and trajectory according to the FOV, changing velocity of movement according to the amount of zoom, closeness to an obstacle or stage in a procedure, and any combination thereof. Preferably, a rule-based approach will be used to determine movement or changes thereof.

In some embodiments, feedback is used to improve general robot accuracy. Feedback can be from operator movements, from image analysis (such as by TRX, ALFX and any combination thereof), from robot movements, and any combination thereof. Preferably, feedback enables closed-loop control of devices in the system, and enables more precise and more accurate control of robotic devices.

In some embodiments, at least one of the devices controllable by the system is bed-mounted. In preferred embodiments, this reduces the footprint of the system over the patient.

In some embodiments, the system comprises system control of at least a portion of an endoscope. In some variants of these embodiments, the endoscope has a wide-angle lens, preferably a high-definition lens. In some variants of these embodiments, the endoscope is an articulated laparoscope; the system can comprise both a wide angle-lens and an articulated endoscope. In some embodiments, with a wide-angle lens, the displayed field of view can be controlled by movement of the endoscope, by virtual FOV control (computer control of the FOV by altering the displayed portion of the image), and any combination thereof. In some embodiments, at least one tool can be automatically tracked by the system.

In preferred embodiments, there is full automation of the control of the at least one robot arm positioning at least one surgical tool in at least two degrees of freedom, and preferably in all 7 degrees of freedom.

In some embodiments, the at least one robotic arm is a snake-like robotic arm

In some embodiments, full control of the at least one robot arm is provided by visual servoing (adaptive control via image analytics). This enables closed-loop control of all DOF's and, therefore, closed loop control of locating a target. Closed loop control also enables optimization by building an adaptive kinematic model for control of the at least one robotic arm.

In embodiments with closed-loop control of robotic movement, lower cost components can be used, such as lower-cost gears, as image-based control (or image manipulation, i.e. moving the image artificially) enables the system to correct for backlash in gear trains in real time, thereby obviating the need to design systems with minimal backlash.

Locations on or in objects, locations on items, and points in the space of the surgical field can be identified as “fixed points” and can be marked. In other words, a 3D point in space can be identified as a known point. The fixed points can be used as locators or identifiers for surgical procedures. For example, a robotic manipulator can move a surgical tool along a path indicated by at least two fixed points, where the path can be, but need not be, a straight line. The surgical tool can be operated along the path, or it can be operated while being moved along the path.

For non-limiting example, fixed points can mark the beginning and end of a path which is a suture line for a suturing procedure. A fixed point can also indicate another location of importance in a surgical field, such as a location for a suture, a locations for a grasper or swab, a location of a suspected lesion, a location of a blood vessel, a location of a nerve, a location of a portion of an organ, and any combination thereof.

Non-limiting examples of the means by which an operator can mark a fixed point include: touching the desired point on a touchscreen, touching its location in a 3D image, moving a marker until the marker coincides with the desired point, touching the desired point with a tool, any other conventional means of identifying a desired point and any combination thereof.

In some embodiments, a label in an image can identify a fixed point. The label can be, for non-limiting example, a number, a shape, a colored region, a textured region, and any combination thereof. The shape can be, for non-limiting example, an arrow, a circle, a square, a triangle, a regular polygon, an irregular polygon, a star, and any combination thereof. A texture can be, for non-limiting example, a parallel lines, dots, a region within which the intensity of a color changes, an area within which the transparency of the overlay changes, and any other conventional means of indicating a texture in a visual field.

In preferred embodiments, the system can be in communication with other devices or systems. In some embodiments, for non-limiting example, the AI-based control software can control at least one surgical tool. In some embodiments, it can be in communication with other advanced imaging systems. In some embodiments, it can function as part of an integrated operating room, by being in communication with such items as, for non-limiting example, other robotic controllers, database systems, bed position controllers, alerting systems, either alerting personnel of possible problems or alerting personnel of equipment (such as tools or supplies) likely to be needed in the near future), automatic tool-supply systems and any combination thereof.

In some embodiments, the AI-based software can have full connectivity with a member of an external information group consisting of: digital documentation, PACS, navigation, other health IT systems, and any combination thereof. This connectivity enables the system to both receive information and to store information in real time.

The received information can be, for non-limiting example, a member of a group consisting of: information about how an operator or an operating team carried out at least one procedure or at least a portion thereof during at least one previous procedure; information about how an operator or operating team responded to an unexpected occurrence (for non-limiting example, severing a blood vessel during removal of a tumor, failure or partial failure of a tool, or slippage or failure of a suture); information about how the patient reacted during at least one previous procedure; information about how at least one other patient reacted during at least one previous procedure, information about how the patient reacted to medication during at least one previous procedure; information about how at least one other patient reacted to medication during at least one previous procedure; and any combination thereof. Such information can be used to alert an operator to a possible adverse reaction, recommend an alternative procedure or medication, suggest an autonomic procedure, automatically execute an autonomic procedure, suggest an alternative autonomic procedure, automatically substitute an alternative autonomic procedure, provide a warning, the name of a surgeon, the name of a member of the operating team, the patient's vital signs during at least one previous procedure, and any combination thereof. The AI-based software can also combine information from one or more sources in order to derive its recommendations or actions.

Vital signs can include, but are not limited to, blood pressure, skin temperature, body temperature, heart rate, respiration rate, blood oxygen, blood CO₂, blood pH, blood hemoglobin, other blood chemical levels, skin color, tissue color, any changes in any of the above, and any combination thereof. Also, predicted changes in any of the above can be received, so that deviations from the predicted changes can be identified and, in some embodiments, presented as an alert to an operator and, in other some embodiments, responded to autonomically by the system.

Information that can be exported to or shared with the external information group can be, for non-limiting example, the procedure which is executed, the patient's vital signs during a procedure, the name of the surgeon, the name of a member of an operating team, the actions of the operator during the procedure, the actions of a member of the operating team during the procedure, the type of autonomic procedure executed, the type of assisting procedure automatically executed (such as, for non-limiting example, maneuvering a laparoscope to provide optimum viewing during a suturing procedure), differences between the actions of the operator during a procedure and during at least one previous procedure (either by the same operator or a different operator), differences between the actions of another member of the operating team during a procedure and during at least one previous procedure (either by the same member of the operating team or a different member of the operating team), difference between a patient's reactions during a procedure to those of the same patient or another patient during at least one previous procedure, and any combination thereof.

At least a portion of at least one procedure can be recorded. A procedure can be edited so that at least one shorter portion, typically a surgical task or an identifiable unit, can be stored, viewed and any combination thereof. At least one stored record of at least one procedure, preferably in 3D, can become part of at least one “big data” analysis, A big data analysis can be, for non-limiting example, for an individual operator, for a hospital or medical center, for a tool, for a robotic maneuvering system and any combination thereof. A recorded procedure can be tagged with at least one identifier, to enhance and simplify searching libraries of stored procedures.

An identifier can include, but is not limited to, an identifier of an operator, type of procedure, a previous procedure during a surgical operation, a parameter, an identifier for an operating room, a physical characteristic of an operating room (e.g., temperature, humidity, type of lighting, time and date of cleaning, cleaning procedure, cleaning materials, type of lighting), a date of the procedure, a time and day of the week of a procedure, a duration of a procedure, a time from start of a previous procedure until start of a procedure, a time from end of a procedure until start of a subsequent procedure, an identifier of a patient, a physical characteristic of a patient, an outcome of a procedure, a type of malfunction during a procedure, severity of malfunction during a procedure, start time of malfunction, end time of malfunction, a general datum, and any combination thereof.

Non-limiting physical characteristics of a patient include: age, height, weight, body mass index, health status, medical status, physical parameter of a patient and any combination thereof.

A physical parameter of a patient can be selected from a group consisting of: health status, blood pressure, heart rate, blood gasses, blood volume, blood hemoglobin, breathing rate, breath depth, EEG, ECG, sweating, and any combination thereof,

A datum from a patient's medical history can be selected from a group consisting of: an illness, an outcome of an illness, a previous procedure, an outcome of a previous procedure, a genetic factor, an effect on said patient of said genetic factor, a predicted effect on said patient of said genetic factor, a medical treatment, an allergy, a medical condition, a psychological factor, and any combination thereof, an outcome of a procedure, length of hospital stay for a patient, a readmission for a patient, a medical treatment for a patient, a subsequent procedure, number of subsequent procedures carried out an operator, a general datum, and any combination thereof.

An outcome can be selected from a group consisting of: a successful aspect, a partially successful aspect, a partial failure in an aspect, a complete failure in an aspect, and any combination thereof.

An aspect is selected from a group consisting of: a complication during a procedure, a complication during another procedure, a component where recovery is smooth and uncomplicated, a rate of recovery from a procedure, a rate of recovery from a complication, a long-term effect of a procedure, a long-term effect of a complication, amount of bleeding during a procedure, amount of bleeding during another procedure, return of an abnormality, speed of healing, an adhesion, patient discomfort, and any combination thereof.

A general datum is selected from a group consisting of: an image of at least a portion of a surgical field, an identifier of an operator, a rating for an operator, a physical characteristic of a patient, a physical characteristic of an operating room, an identifier of a procedure, type of procedure, time of a beginning of a procedure, time of an intermediate point of a procedure, time of an end point of a procedure, duration of a procedure, time between end of a procedure and beginning of another procedure, time of creation of a critical point, location of a critical point, time of creation of a fixed point, location of a fixed point, a medication, a medical device, an identifier of a surgical object, a type of a surgical object, a number used for a surgical object, a cleaning status for a surgical object, a comment, a parameter, a metric, occurrence of a malfunction, severity of a malfunction, start time of a malfunction, end time of a malfunction, reason for start of a malfunction, reason for end of a malfunction, occurrence of an adverse event, a test, an image from another modality, an overlay, a label, a note, and any combination thereof.

A parameter is selected from a group consisting of: 2D position of at least a portion of at least one item, 2D orientation of at least a portion of at least one item, 3D position of at least a portion of at least one item, 3D orientation of at least a portion of at least one item, 2D projection of a 3D position of at least a portion of said at least one item, movement of at least a portion of at least one said item, energy use; idle time, approach time, speed, maximum speed, speed profile, acceleration, motion smoothness, path length, distance efficiency, depth perception, transit profile, deviation on horizontal plane, deviation on vertical plane, response orientation, economy of area (EOA), economy of volume (EOV), number of movements, force range, interquartile force range, integral of the force, integral of the grasping force, integral of the Cartesian force, first derivative of the force, second derivative of the force, third derivative of the force, lighting level, amount of suction, amount of fluid flow, heating level in an ablator, amount of defogging, amount of smoke removal, activation of an item, deactivation of an item, bleeding, change in heart rate, change in blood pressure, change in color of an organ,

A medical device can be selected from a group consisting of: a heating blanket, a pump, a catheter, and any combination thereof.

Occurrence of an adverse event can be selected from a group consisting of: unexpected bleeding, undesirable change in blood pressure, undesirable change in heart rate, undesirable change in consciousness state, pain, and any combination thereof.

A medication can be selected from a group consisting of: an antibiotic, an anesthetic, plasma, blood, saline, coagulant, anticoagulant, blood pressure medication, heart medication, and any combination thereof.

A medical treatment can be selected from a group consisting of: administering a medication, applying a medical device, prescribing a course of exercise, administering physiotherapy, and any combination thereof.

A test can be selected from a group consisting of: a blood test, a blood pressure measurement, an EEG, and ECG, and any combination thereof.

Another modality can be selected from a group consisting of: MRI, CT, ultrasound, X-ray, fluorography, fluoroscopy, molecular imaging, scintigraphy, SPECT, positron emission tomography (PET), other types of tomography, elastography, tactile imaging, photoacoustic imaging, thermography, functional near-infrared spectroscopy (FNIR) and any combination thereof.

An image from another modality can be stored or real-time.

A note, a comment and any combination thereof can be selected from a group consisting of: a descriptor of a previously-performed procedure, a list of at least one previously performed procedure, how a procedure was executed, why a procedure was chosen, an assessment of a patient, a prediction, an item to be added to a medical history, a method of executing a procedure, and any combination thereof.

A critical point can be selected from a group consisting of: a location in said surgical field, a beginning of a procedure, an end of a procedure, an intermediate point in a procedure and any combination thereof.

At least one image of at least a portion of a surgical field, a second modality image and any combination thereof can be selected from a group consisting of: a 2D image, a 3D image, a panoramic image, a high resolution image, a 3D image reconstructed from at least one 2D image, a 3D stereo image, and any combination thereof.

Tagging, supplying an identifier, can be manual or automatic. For non-limiting example, typically, an identifier of an operator will be entered manually. In another non-limiting example, a critical point or a fixed point can be tagged manually or automatically. For non-limiting example, manual tagging can be by an operator indicating, by word, by gesture, or by touching a touchscreen, that a given point, such as the current position of a surgical object, is to be tagged as a critical point or a fixed point. For non-limiting example, automatic tagging can occur when a system identifies a point as a critical point or a fixed point.

It should be emphasized that it is within the scope of the present invention wherein assessment of quality of functioning for at least one surgical object includes the additional information which can be obtained from an accelerometer, a motion sensor, an IMU, a sensor wearable by an operator, a sensor attachable to a surgical object, an RFID tag attachable to a surgical object, the state of a surgical object, an ultrasound sensor, an infrared sensor, a CT image, an MRI image, and X-ray image, a gyroscope, a tachometer, a shaft encoder, a rotary encoder, a strain gauge and any combination thereof.

It should be noted that any combination of the above embodiments also comprises an embodiment of the system.

1-86. (canceled)

87. A system for identifying at least one surgical procedure, comprising:
at least one robotic manipulator connectable to said at least one surgical tool;
at least one imaging device configured to real time provide at least one image in a field of view of a surgical environment;
at least one processor in communication with said robotic manipulator and said imaging device; said processor is configured to control maneuvering of said at least one surgical tool by said robotic manipulator; said at least one processor is configured to (i) analyze at least one image from said at least one imaging device and, (ii) to identify from said at least one image at least one spatial position of at least one item, SPᵢₜₑₘ; and,
at least one communicable database configured to (i) store said at least one surgical procedure; said at least one surgical procedure is characterized by at least one spatial position of at least one item SP_{stored}; (ii) real-time store at least one of said spatial position, SPᵢₜₑₘ, of at least one said item;
wherein said at least one processor is configured to identify at least one said surgical procedure being performed by identifying at least partial match between at least one of said SPᵢₜₑₘ and at least one of said SP_{stored}.

88. The system of claim 87, wherein at least one of the following is true:
a. said item is selected from a group consisting of: said at least one surgical tool, a light source, a blood vessel, an organ, a nerve, and a ligament, a lesion, a tumor, smoke, fluid flow, bleeding, a fixed point, a critical point, and any combination thereof;
b. said spatial position is selected from a group consisting of: a 2D position of at least a portion of the object; a 2D orientation of at least a portion of the object; a 3D position of at least a portion of the object; a 3D orientation of at least a portion of the object; a 2D projection of a 3D position of at least a portion of the object; and any combination thereof;
c. said procedure is initiatable by a member of a group consisting of: manually by a command from an operator, automatically by a command from said processor and any combination thereof;
d. said procedure is identifiable from at least two fixed points, said procedure comprising a member of a group consisting of: maneuvering said robotic manipulator along a path joining said at least two fixed points, controlling operation of at least one said surgical tool and any combination thereof;
e. said database is configured to store a member of a group consisting of: an autonomically-executed procedure, an automatically-executed procedure, a manually executed procedure and any combination thereof; and
f. said selection of said at least one surgical procedure is at least partially based on at least one procedure-related datum;
g. said system is additionally configured to control execution of said procedure by maneuvering said at least one robotic manipulator;
h. said system is additionally configured to accept definition of a location for a member of a group consisting of: said fixed point, said critical point and any combination thereof, and to store said location; and
i. said system further comprises a manual override, said procedure stoppable by means of said manual override.

89. The system of claim 87, wherein said processor is additionally configured to control operation of at least one second surgical tool, at least one of the following being true:
a. said control of operation of said at least one second surgical tool is autonomic control;
b. said at least one second surgical tool is selected from a group consisting of: a laparoscope, an endoscope, an ablator, a fluid supply mechanism, a retractor, a grasper, a suturing mechanism, a pair of tweezers, a forceps, a light source, a vacuum source, a suction device, and any combination thereof; and
c. said second surgical tool is said surgical tool.

90. The system of claim 87, additionally configured to provide a message, said message configured to provide information, said message being selected from a group consisting of: an audible message, a visual message, a tactile message and any combination thereof; at least one of the following being true:
a. said visual message is selected from a group consisting of: a constant-brightness light, a variable-brightness light, a constant-color light, a variable-color light, a patterned light and any combination thereof;
b. said audible message is selected from a group consisting of: a constant-loudness sound, a variable-loudness sound, a constant-pitch sound, a variable-pitch sound, a patterned sound and any combination thereof; and
c. said tactile message is selected from a group consisting of: a vibration, a stationary pressure, a moving pressure and any combination thereof.

91. The system of claim 87, wherein at least one record is selectable based upon an identifier; said database is configured to store said identifier, said identifier is selected from a group consisting of: an identifier of an operator, an identifier of an operating room, a physical characteristic of an operating room, a number of times said operator has executed a procedure, a physical characteristic of an operating room, a date of said procedure, a time of said procedure, a duration of said procedure, a vital sign of a patient, an identifier of a patient, a physical characteristic of a patient, an outcome of a procedure, length of hospital stay for a patient, a readmission for a patient a datum from a patient's medical history, number of said at least one procedures carried out by an operator, cleaning status of an operating room, a general datum, and any combination thereof.

92. The system of claim 91, wherein at least one of the following is true:
a. said cleaning status of an operating room is selected from a group consisting of: time of last cleaning, date of last cleaning, cleaning procedure, cleaning material and any combination thereof; and
b. said identifier is storable as a function of time.

93. The system of claim 91, wherein said outcome is selected from a group consisting of: a successful aspect, a partially successful aspect, a partial failure in an aspect, a complete failure in an aspect, and any combination thereof said aspect being selected from a group consisting of: a complication during a procedure, a complication during another procedure, a component where recovery is smooth and uncomplicated, a rate of recovery from a procedure, a rate of recovery from a complication, a long-term effect of a procedure, a long-term effect of a complication, amount of bleeding during a procedure, amount of bleeding during another procedure, return of an abnormality, speed of healing, an adhesion, patient discomfort, and any combination thereof.

94. The system of claim 91, wherein said datum from a patient's medical history is selected from a group consisting of: an illness, an outcome of an illness, a previous procedure, an outcome of a previous procedure, a genetic factor, an effect on said patient of said genetic factor, a predicted effect on said patient of said genetic factor, a medical treatment, an allergy, a medical condition, a psychological factor, and any combination thereof, an outcome of a procedure, length of hospital stay for a patient, a readmission for a patient, a medical treatment for a patient, a subsequent procedure, number of subsequent procedures carried out an operator, a general datum, and any combination thereof.

95. The system of claim 94, wherein said general datum is selected from a group consisting of: an image of at least a portion of a surgical field, an identifier of an operator, a rating for an operator, a physical characteristic of a patient, a physical characteristic of an operating room, an identifier of a procedure, type of procedure, time of a beginning of a procedure, time of an intermediate point of a procedure, time of an end point of a procedure, duration of a procedure, time between end of a procedure and beginning of another procedure, time of creation of a critical point, location of a critical point, time of creation of a fixed point, location of a fixed point, a medication, a medical device, an identifier of a surgical object, a type of a surgical object, a number used for a surgical object, a cleaning status for a surgical object, a comment, a parameter, a metric, occurrence of a malfunction, severity of a malfunction, start time of a malfunction, end time of a malfunction, reason for start of a malfunction, reason for end of a malfunction, occurrence of an adverse event, a test, an image from another modality, an overlay, a label, a note, and any combination thereof at least one of the following being true:
a. said parameter is selected from a group consisting of: 2D position of at least a portion of at least one item, 2D orientation of at least a portion of at least one item, 3D position of at least a portion of at least one item, 3D orientation of at least a portion of at least one item, 2D projection of a 3D position of at least a portion of said at least one item, movement of at least a portion of at least one said item, energy use; idle time, approach time, speed, maximum speed, speed profile, acceleration, motion smoothness, path length, distance efficiency, depth perception, transit profile, deviation on horizontal plane, deviation on vertical plane, response orientation, economy of area (EOA), economy of volume (EOV), number of movements, force range, interquartile force range, integral of the force, integral of the grasping force, integral of the Cartesian force, first derivative of the force, second derivative of the force, third derivative of the force, lighting level, amount of suction, amount of fluid flow, heating level in an ablator, amount of defogging, amount of smoke removal, activation of an item, deactivation of an item, bleeding, change in heart rate, change in blood pressure, change in color of an organ, and any combination thereof;
b. said physical characteristic of an operating room is selected from a group consisting of: temperature, humidity, type of lighting, and any combination thereof,
c. said medical device is selected from a group consisting of: a heating blanket, a pump, a catheter, and any combination thereof;
d. said occurrence of an adverse event is selected from a group consisting of: unexpected bleeding, undesirable change in blood pressure, undesirable change in heart rate, undesirable change in consciousness state, pain, and any combination thereof;
e. said physical characteristic of said patient is selected from a group consisting of: age, height, weight, body mass index, physical parameter of said patient, and any combination thereof, said physical parameter of said patient being selected from a group consisting of: health status, blood pressure, heart rate, blood gasses, blood volume, blood hemoglobin, breathing rate, breath depth, EEG, ECG, sweating, and any combination thereof,
f. said medication is selected from a group consisting of: an antibiotic, an anesthetic, plasma, blood, saline, coagulant, anticoagulant, blood pressure medication, heart medication, and any combination thereof,
g. said medical treatment is selected from a group consisting of: administering a medication, applying a medical device, prescribing a course of exercise, administering physiotherapy, and any combination thereof,
h. said test is selected from a group consisting of: a blood test, a blood pressure measurement, an EEG, and ECG, and any combination thereof;
i. said other modality is selected from a group consisting of: MRI, CT, ultrasound, X-ray, fluorography, fluoroscopy, molecular imaging, scintigraphy, SPECT, positron emission tomography (PET), other types of tomography, elastography, tactile imaging, photoacoustic imaging, thermography, functional near-infrared spectroscopy (FNIR) and any combination thereof;
j. said image from said other modality can be stored or real-time;
k. a member of a group consisting of said note, said comment and any combination thereof is selected from a group consisting of: a descriptor of a previously-performed procedure, a list of at least one previously performed procedure, how a procedure was executed, why a procedure was chosen, an assessment of a patient, a prediction, an item to be added to a medical history, a method of executing a procedure, and any combination thereof;
l. The system of claim 24, wherein said critical point is selected from a group consisting of: a location in said surgical field, a beginning of a procedure, an end of a procedure, an intermediate point in a procedure and any combination thereof; and
m. a member of a group consisting of said at least one image of at least a portion of a surgical field, said second modality image and any combination thereof is selected from a group consisting of: a 2D image, a 3D image, a panoramic image, a high resolution image, a 3D image reconstructed from at least one 2D image, a 3D stereo image, and any combination thereof.

96. The system of claim 87, wherein said system is in at least one-way communication with a member of a group consisting of: digital documentation, PACS, navigation, a health IT system, and any combination thereof said communication comprising communicating procedure-related data selected from a group consisting of: a name of an operator, a name of an assistant, an identifier of an operator, an identifier of an assistant, an identifier of an operating room, a physical characteristic of an operating room, a physical characteristic of an operating room as a function of time, a date of said procedure, a start time of said procedure, an end time of said procedure, a duration of said procedure, a vital sign of a patient, a name of a patient, a physical characteristic of a patient, an outcome of said procedure, length of hospital stay for a patient, a readmission for a patient, a number of times said operator has executed a procedure, a date of a previous procedure, a start time of a previous procedure, an end time of a previous procedure, a duration of a previous procedure, a vital sign of a previous patient, a name of a previous patient, a physical characteristic of a previous patient, length of hospital stay for a previous patient, a readmission for a previous patient, and any combination thereof.

97. A method for identifying at least one surgical procedure, comprising steps of:
providing a system for identifying at least one surgical procedure comprising:
at least one robotic manipulator connectable to said at least one surgical tool;
at least one imaging device configured to real time provide at least one image in a field of view of a surgical environment;
at least one processor in communication with said robotic manipulator and said imaging device, said processor is configured to control maneuvering of said at least one surgical tool by said robotic manipulator; said at least one processor is configured to (i) analyze at least one image from said at least one imaging device and (ii) identify from said at least one image at least one spatial position of at least one item, SPᵢₜₑₘ; and
at least one communicable database configured to (i) store at least one surgical procedure; said at least one surgical procedure is characterized by at least one spatial position of at least one item SP stored; stored; (ii) real-time store at least one said spatial position, SPᵢₜₑₘ, of at least one said item;
connecting said at least one surgical tool to said robotic manipulator;
acquiring, via said imaging device, at least one said image of said field of view;
analyzing said at least one image and identifying, from said analysis, said at least one spatial position of said at least one item, SPᵢₜₑₘ;
real-time storing at least one said spatial position of at least one said item, SPᵢₜₑₘ; and
identifying at least one said surgical procedure being performed by identifying at least partial match between at least one of said SPᵢₜₑₘ and at least one of said SP_{stored}.

98. The method of claim 97, additionally comprising at least one of the following steps:
a. selecting said item from a group consisting of: said at least one surgical tool, a light source, a blood vessel, an organ, a nerve, and a ligament, a lesion, a tumor, smoke, fluid flow, bleeding, a fixed point, a critical point, and any combination thereof;
b. selecting said spatial position from a group consisting of: a 2D position of at least a portion of the object; a 2D orientation of at least a portion of the object; a 3D position of at least a portion of the object; a 3D orientation of at least a portion of the object; a 2D projection of a 3D position of at least a portion of the object; and any combination thereof;
c. initiating said procedure by a member of a group consisting of: manually by a command from an operator, automatically by a command from said processor and any combination thereof;
d. identifying said procedure from at least two fixed points, said procedure comprising a member of a group consisting of: maneuvering said robotic manipulator along a path joining said at least two fixed points, controlling operation of at least one said surgical tool along a path joining said at least two fixed points and any combination thereof;
e. storing in said database a member of a group consisting of: an autonomically-executed procedure, an automatically-executed procedure, a manually executed procedure and any combination thereof;
f. selecting said at least one surgical procedure at least partially based on at least one procedure-related datum;
g. controlling execution of said at least one procedure by maneuvering said at least one robotic manipulator;
h. accepting definition of a location of a member of a group consisting of: said fixed point, said critical point and any combination thereof, and of storing said location; and
i. providing a manual override, and of stopping said procedure by means of said manual override.

99. The method of claim 97, additionally comprising step of controlling operation of at least one second surgical tool; and additionally comprising at least one of the following steps:
a. autonomically controlling operation of said at least one second surgical tool;
b. selecting said at least one second surgical tool from a group consisting of: a laparoscope, an endoscope, a suction device, a vacuum source, a light source, an ablator, a fluid supply mechanism, a retractor, a grasper, a suturing mechanism, a pair of tweezers, a pair of tweezers, a forceps, and any combination thereof; and
c. selecting second surgical tool to be said surgical tool.

100. The method of claim 97, additionally comprising steps of providing a message, said message configured to provide information; and selecting said message from a group consisting of: an audible message, a visual message, a tactile message and any combination thereof, and additionally comprising at least one of the following steps:
a. selecting said visual message from a group consisting of: a constant-brightness light, a variable-brightness light, a constant-color light, a variable-color light, a patterned light and any combination thereof,
b. selecting said audible message from a group consisting of: a constant-loudness sound, a variable-loudness sound, a constant-pitch sound, a variable-pitch sound, a patterned sound and any combination thereof; and
c. selecting said tactile message from a group consisting of: a vibration, a stationary pressure, a moving pressure and any combination thereof.

101. The method of claim 97, additionally comprising steps of selecting at least one record based upon an identifier; and selecting said identifier from a group consisting of: an identifier of an operator, an identifier of an operating room, a physical characteristic of an operating room, a number of times said operator has executed a procedure, a physical characteristic of an operating room, a date of said procedure, a time of said procedure, a duration of said procedure, a vital sign of a patient, an identifier of a patient, a physical characteristic of a patient, an outcome of a procedure, length of hospital stay for a patient, a readmission for a patient a datum from a patient's medical history, number of said at least one procedures carried out by an operator, cleaning status of an operating room, a general datum, and any combination thereof, and of storing in said database and of storing said identifier in said database.

102. The method of claim 101, additionally comprising at least one of the following steps:
a. selecting said cleaning status of an operating room from a group consisting of: time of last cleaning, date of last cleaning, cleaning procedure, cleaning material and any combination thereof; and
b. storing said identifier as a function of time.

103. The method of claim 101, additionally comprising steps of selecting said outcome from a group consisting of: a successful aspect, a partially successful aspect, a partial failure in an aspect, a complete failure in an aspect, and any combination thereof, and of selecting said aspect from a group consisting of: a complication during a procedure, a complication during another procedure, a component where recovery is smooth and uncomplicated, a rate of recovery from a procedure, a rate of recovery from a complication, a long-term effect of a procedure, a long-term effect of a complication, amount of bleeding during a procedure, amount of bleeding during another procedure, return of an abnormality, speed of healing, an adhesion, patient discomfort, and any combination thereof.

104. The method of claim 101, additionally comprising step of selecting said datum from a patient's medical history from a group consisting of: an illness, an outcome of an illness, a previous procedure, an outcome of a previous procedure, a genetic factor, an effect on said patient of said genetic factor, a predicted effect on said patient of said genetic factor, a medical treatment, an allergy, a medical condition, a psychological factor, and any combination thereof, an outcome of a procedure, length of hospital stay for a patient, a readmission for a patient, a medical treatment for a patient, a subsequent procedure, number of subsequent procedures carried out an operator, a general datum, and any combination thereof.

105. The method of claim 104, additionally comprising step of selecting said general datum from a group consisting of: an image of at least a portion of a surgical field, an identifier of an operator, a rating for an operator, a physical characteristic of a patient, a physical characteristic of an operating room, an identifier of a procedure, type of procedure, time of a beginning of a procedure, time of an intermediate point of a procedure, time of an end point of a procedure, duration of a procedure, time between end of a procedure and beginning of another procedure, time of creation of a critical point, location of a critical point, time of creation of a fixed point, location of a fixed point, a medication, a medical device, an identifier of a surgical object, a type of a surgical object, a number used for a surgical object, a cleaning status for a surgical object, a comment, a parameter, a metric, occurrence of a malfunction, severity of a malfunction, start time of a malfunction, end time of a malfunction, reason for start of a malfunction, reason for end of a malfunction, occurrence of an adverse event, a test, an image from another modality, an overlay, a label, a note, and any combination thereof; and comprising at least one of the following steps:
a. selecting said parameter from a group consisting of: 2D position of at least a portion of at least one item, 2D orientation of at least a portion of at least one item, 3D position of at least a portion of at least one item, 3D orientation of at least a portion of at least one item, 2D projection of a 3D position of at least a portion of said at least one item, movement of at least a portion of at least one said item, energy use; idle time, approach time, speed, maximum speed, speed profile, acceleration, motion smoothness, path length, distance efficiency, depth perception, transit profile, deviation on horizontal plane, deviation on vertical plane, response orientation, economy of area (EOA), economy of volume (EOV), number of movements, force range, interquartile force range, integral of the force, integral of the grasping force, integral of the Cartesian force, first derivative of the force, second derivative of the force, third derivative of the force, lighting level, amount of suction, amount of fluid flow, heating level in an ablator, amount of defogging, amount of smoke removal, activation of an item, deactivation of an item, bleeding, change in heart rate, change in blood pressure, change in color of an organ, and any combination thereof;
b. selecting said physical characteristic of an operating room from a group consisting of: temperature, humidity, type of lighting, and any combination thereof;
c. selecting said medical device is selected from a group consisting of: a heating blanket, a pump, a catheter, and any combination thereof;
d. wherein said occurrence of an adverse event from a group consisting of: unexpected bleeding, undesirable change in blood pressure, undesirable change in heart rate, undesirable change in consciousness state, pain, and any combination thereof;
e. selecting said physical characteristic of said patient from a group consisting of: age, height, weight, body mass index, physical parameter of said patient, and any combination thereof;
f. selecting said physical parameter of said patient from a group consisting of: health status, blood pressure, heart rate, blood gasses, blood volume, blood hemoglobin, breathing rate, breath depth, EEG, ECG, sweating, and any combination thereof;
g. selecting said medication from a group consisting of: an antibiotic, an anesthetic, plasma, blood, saline, coagulant, anticoagulant, blood pressure medication, heart medication, and any combination thereof;
h. selecting said medical treatment from a group consisting of: administering a medication, applying a medical device, prescribing a course of exercise, administering physiotherapy, and any combination thereof;
i. selecting said test from a group consisting of: a blood test, a blood pressure measurement, an EEG, and ECG, and any combination thereof;
j. selecting said other modality from a group consisting of: MRI, CT, ultrasound, X-ray, fluorography, fluoroscopy, molecular imaging, scintigraphy, SPECT, positron emission tomography (PET), other types of tomography, elastography, tactile imaging, photoacoustic imaging, thermography, functional near-infrared spectroscopy (FNIR) and any combination thereof;
k. providing said image from said other modality either stored or real-time;
l. selecting a member of a group consisting of said note, said comment and any combination thereof from a group consisting of: a descriptor of a previously-performed procedure, a list of at least one previously performed procedure, how a procedure was executed, why a procedure was chosen, an assessment of a patient, a prediction, an item to be added to a medical history, a method of executing a procedure, and any combination thereof;
m. selecting said critical point from a group consisting of: a location in said surgical field, a beginning of a procedure, an end of a procedure, an intermediate point in a procedure and any combination thereof; and
n. selecting a member of a group consisting of said at least one image of at least a portion of a surgical field, said second modality image and any combination thereof from a group consisting of: a 2D image, a 3D image, a panoramic image, a high resolution image, a 3D image reconstructed from at least one 2D image, a 3D stereo image, and any combination thereof.

106. The method of claim 97, additionally comprising steps of providing for said system at least one-way communication with a member of a group consisting of: digital documentation, PACS, navigation, a health IT system, and any combination thereof; and of providing, for said communication, procedure-related data, and of selecting said procedure-related data from a group consisting of: a name of an operator, a name of an assistant, an identifier of an operator, an identifier of an assistant, an identifier of an operating room, a physical characteristic of an operating room, a physical characteristic of an operating room as a function of time, a date of said procedure, a start time of said procedure, an end time of said procedure, a duration of said procedure, a vital sign of a patient, a name of a patient, a physical characteristic of a patient, an outcome of said procedure, length of hospital stay for a patient, a readmission for a patient, a number of times said operator has executed a procedure, a date of a previous procedure, a start time of a previous procedure, an end time of a previous procedure, a duration of a previous procedure, a vital sign of a previous patient, a name of a previous patient, a physical characteristic of a previous patient, length of hospital stay for a previous patient, a readmission for a previous patient, and any combination thereof.
